(19) United States
(12) Patent Application Publication
(10) Pub. No.: US 2010/0196264 A1
(43) Pub. Date: Aug. 5, 2010

(54) USE OF SOMATOSTATIN ANALOGS IN MYOCARDIAL PERFUSION IMAGING

(75) Inventor: Chester Drum, Cambridge, MA (US)

Correspondence Address: EDWARDS ANGELL PALMER & DODGE LLP, P.O. BOX 55874, BOSTON, MA, 02205, US

(73) Assignee: THE BRIGHAM AND WOMEN'S HOSPITAL, INC., Boston, MA (US)

(21) Appl. No.: 12/600,500

(22) PCT Filed: May 15, 2008
(86) PCT No.: PCT/US08/06265

§ 371 Date: Apr. 5, 2010

Related U.S. Application Data
(60) Provisional application No. 60/930,903, filed on May 18, 2007.

(51) Int. Cl. A61K 51/00; A61K 38/08; A61K 49/00; A61P 9/00; A61P 43/00

(52) U.S. Cl. 424/1.11; 514/16; 424/9.1

(57) ABSTRACT

The present invention features inter alia polypeptides comprising an Fc region comprising genetically-fused Fc moieties. In addition, the instant invention provides, e.g., methods for treating or preventing a disease or disorder in subject by administering the binding polypeptides of the invention to said subject.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Coronary artery disease (CAD) is a major determinant of both mortality and morbidity in the United States and throughout the world. More than 13 million Americans suffer from CAD, with 470 thousand deaths attributed to CAD in 2003. Heart Disease and Stroke Statistics-2006 Update. 2006, American Heart Association: Dallas, Tex. (Heart Disease and Stroke Statistics-2006 Update. 2006, American Heart Association: Dallas, Tex. http://www.americanheart.org.)

Myocardial perfusion imaging, (MPI), is an integral part of the modern diagnosis and risk stratification of CAD (Klocke F J, B. M., Bateman T M, Berman D S, Carabello B A, Cerqueira M D, DeMaria A N, Kennedy J W, Lorell B H, Messer J V, O'Gara P T, Russel R O Jr., St. John Sutton, M G, Udelson J E, Verani M S, Williams K A., ACC/AHA/ASNC Guidelines for the Clinical Use of Cardiac Radionuclide Imaging: A report of the American College of Cardiology/American Heart Association Task Force on Practice Guidelines. 2003, American College of Cardiology/American Heart Association: Bethesda, Md. p. 69). This technique involves the infusion of a radioactive tracer compound that, when imaged with a gamma-ray sensitive camera, gives a ‘snapshot’ of the distribution of blood flow within the myocardium at the time of injection. The resultant images help to determine both the functional capacity of coronary arteries in addition to the underlying viability of supplied myocardium. The administration of radiotracer at rest is often accompanied by a second administration during a physiologic or pharmacologic stress. By comparing rest and stress images, areas of the heart with normal, inadequate or no blood flow, (i.e. scar), can be identified (Zipes, D., Libby, P., Bonow, R., Braunwald, E., Braunwald's Heart Disease: A Textbook of Cardiovascular Medicine. 7th ed. 2005, Philadelphia: Elsevier Saunders. 2183). Several varieties of radiotracer are available, including Rb-82, 5-FDG, Tc-99m-sestamibi, Thallium-201 and Tc-99m-tetrophosmin. Types of stresses used in clinical practice include exercise (usually walking or running on a treadmill), adenosine, dipyridamole or dobutamine administration. The particular selection of tracer compound and mode of stress depends on the clinical question being asked and the particular technologies available to the ordering physician.

The utility of myocardial perfusion imaging in the management of cardiovascular disease is without question. However, the full diagnostic potential of the test has yet to be realized due to the regular appearance of artifacts that obscure the interpretation of cardiac images and decrease its diagnostic accuracy. (FIG. 1) One of the most common sources of artifact in MPI is radiotracer uptake by the liver and gut. Specifically, emission of signal from areas in proximity to the heart can confuse radiographic interpretation. (FIG. 2) Anatomically, the inferior aspect of the human heart lies a centimeter or less away from the superior surface of the liver, stomach and occasionally the small bowel. Commercial radiotracers, in particular thallium-201 and Tc-99m labeled agents, are absorbed from the blood by the liver and can enter either the small bowel or reflux into the stomach. For example, in a typical Tc-99m-setsamibi administration at rest, 1.2% of the total dose is taken into the heart, whereas 19% is absorbed into the liver (Cardiolite Product Insert. 2003, Bristol-Myers Squibb, Medical Imaging: N. Billerica, Mass. p. 2).

Due to the deleterious effects of subdiaphragmatic radiotracer uptake on myocardial perfusion imaging, several technologies have been examined in an attempt to reduce artifact signals.

Compared with images taken at rest or while the patient is undergoing a chemical form of cardiac stress, images taken while the patient is undergoing physical exercise have reduced levels of hepatic and gut uptake of radiotracer. (FIG. 3) Although this is a solution for some patients, many patients are unable to undergo exercise stress testing due to issues of decreased mobility, balance and deconditioning. These patients must therefore use a pharmacologic stress such as adenosine or dipyridamole. Also, in many laboratories, patients undergo resting images before the exercise portion of the procedure. In this situation, the rest images retain high relative amounts of hepatic and gut radiotracer uptake, which compounds problems in rest versus stress image comparison (Iskandrian, A. E., Verani, Mario S., Nuclear Cardiac Imaging: Principles and Applications. 2003, New York, N.Y.: Oxford Press. 509).

Additional studies have attempted to increase the anatomic separation between the bottom of the heart and subdiaphragmatic structures through prone positioning during image acquisition (Dogruca, Z., et al., A comparison of Tl-201 stress-reinjection-prone SPECT and Tc-99m-sestamibi gated SPECT in the differentiation of inferior wall defects from artifacts. Nucl Med Commun, 2000. 21(8): p. 719-27; Schoss, R. M. and R. J. Gorten, Comparison of supine versus prone tomographic myocardial imaging. Effect on false-positive rate. Clin Nucl Med, 1996. 21(6): p. 445-51; Kiat, H., et al., Quantitative stress-redistribution thallium-201 SPECT using prone imaging: methodologic development and validation. J Nucl Med, 1992. 33(8): p. 1509-15; Segall, G. M., M. J. Davis, and M. L. Goris, Improved specificity of prone versus supine thallium SPECT imaging. Clin Nucl Med, 1988. 13(12): p. 915-6; Esquerre, J. P., Prone versus supine thallium-201 myocardial SPECT. J Nucl Med, 1989. 30(10): p. 1738-9; Segall, G. M. and M. J. Davis, Prone versus supine thallium myocardial SPECT: a method to decrease artifactual inferior wall defects. J Nucl Med, 1989. 30(4): p. 548-55). Although this has been shown to be helpful with diaphragmatic attenuation, the use of prone positioning is limited due to patient inconvenience and the need to re-image the patient after initial identification of diaphragmatic artifact.

Other studies have focused on the administration of food prior to the exam in order to promote repositioning of the stomach and increased intestinal mobility (Boz, A., et al., The volume effect of the stomach on intestinal activity on same-day exercise—rest Tc-99m tetrofosmin myocardial imaging. Clin Nucl Med, 2001. 26(7): p. 622-5; Boz, A., et al., The effects of solid food in prevention of intestinal activity in Tc-99m tetrofosmin myocardial perfusion scintigraphy. J Nucl Cardiol, 2003. 10(2): p. 161-7; Hurwitz, G. A., et al., Investigation of measures to reduce interfering abdominal activity on rest myocardial images with Tc-99m sestamibi. Clin Nucl Med, 1993. 18(9): p. 735-41; van Dongen, A. J. and P. P. van Rijk, Minimizing liver, bowel, and gastric activity in myocardial perfusion SPECT. J Nucl Med, 2000. 41(8): p. 1315-7. Unfortunately, these methods have not demonstrated a clinically useful benefit. A pharmacological approach to promoting gut motility was examined using the drug metoclopromide. Similar to preprocedural feeding, metoclopromide did not significantly decrease the obscuring effect of abdominal radiotracer uptake (Weinmann, P. and J. L. Moretti, Metoclopramide has no effect on abdominal activity of sestamibi in myocardial SPET. Nucl Med Commun, 1999. 20(7): p. 623-5). Likewise, the gastric stimulant, cholecystokinin, has also been evaluated without success (Middleton, G. W. and J. H. Williams, Significant gastric reflux of technetium-99m-MIBI in SPECT myocardial imaging. J Nucl Med, 1994. 35(4): p. 619-20). Lastly, the use of aminophyllin to counter dipyridamole stress does not prevent artifacts from subsequent images taken after the dipyridamole injection (Yuksel, M., et al., The effect of aminophylline administration on 99 mTc-MIBI lung and liver uptake in patients with or without myocardial ischemia. Rev Esp Med Nucl, 2000. 19(6): p. 423-7)

Thus, there remains a need to reduce uptake of radiotracer during myocardial perfusion imaging that is effective during the acquisition of both resting images and stress images and reduces the obscuring effect of abdominal radiotracer uptake for image analysis.

### SUMMARY OF THE INVENTION

The present invention is based, at least in part, on the discovery that somatostatin can reduce extracardiac accumulation of a radiotracer during myocardial perfusion imaging. Thus, in one aspect, the invention provides a method for reducing extracardiac accumulation of a radiotracer during myocardial perfusion imaging in a subject comprising administering to a subject in need of myocardial perfusion imaging an effective amount of somatostatin or one or more somatostatin analogs or a physiologically acceptable salt thereof, thereby reducing extracardiac accumulation of a radiotracer during myocardial perfusion imaging

In one embodiment, the method comprises administering one or more somatostatin analogs or a physiologically acceptable salt thereof.

In another embodiment, the somatostatin analog is prosomatostatin, somatostatin-28, somatostatin-14, octreotide, octreotide acetate, lanreotide, seglitide, vapreotide, AN-238, SMS 201-995, SDZ CO 611, RC 160, SMS-D70, SOM 230, KE 108, CGP 23996, BIM 23014, L362,855, L054,522, a cyclic peptide having somatostatin properties, a cyclohexapeptide having somatostatin agonist properties, an octopeptide having somatostatin agonist properties or a small molecule that mimics the pharmacological properties of somatostatin; more preferably the somatostatin analog is octreotide, octreotide acetate, or lanreotide. In yet another embodiment, the somatostatin analog is a radio labeled somatostatin or a radio labeled peptide analog of somatostatin. In still another distinct embodiment, the somatostatin analog is not a radio labeled somatostatin or a radio labeled peptide analog of somatostatin. In further embodiment, one or more additional somatostatin analogs or physiologically acceptable salts thereof may also be administered to the subject.

In one embodiment, the radiotracer whose uptake is being prevented is Albumin aggregated iodinated I 131 serum, Albumin chromated Cr 51 serum, Albumin iodinated I 125 serum, Albumin iodinated I 131 serum, Ammonia N 13, Carbon monoxide C 11, Carbon C 14 urea, Chromic phosphate P 32, Cyanocobalamin Co 57, Cyanocobalamin Co 58/Co 57, Ferrous citrate Fe 59, Fibrinogen 1 125, Fludeoxyglucose F 18 Fluorodopa F 18, Gallium citrate Ga 67, I 131 radio labeled B1 monoclonal antibody, Indium in 111 capromab pendetide, Indium in 111 chloride, Indium in 111 imciromab pentetate, Indium in 111 immune globulin intravenous pentetas, Indium in 111 oxyquinoline, Indium in 111 pentetate, Indium in 111 pentetreotide, Indium in 111 satumomab pencletidee, lobenguane sulfate I 123, lobenguane sulfate I 131, locanlidic acid I 123, Iodocholesterol I 131, Iodohippurate sodium I 123, Iodohippurate sodium I 131, Iodomethylnorcholesterol I 131, lofetamine hydrochloride I 123, lothalamate sodium I 125, Krypton Kr 81m, Mesiperone C 11, Methionine C 11, Raclopride C 11, Rhenium Re 186 etidronate, Rubidium chloride Rb 82, Samarium Sm 153 lexidronam pentasodium, Selenomethionine Se 75, Sodium acetate C 11, Sodium chromate Cr 51, Sodium fluoride F 18, Sodium iodide I 123, Sodium iodide I 131, Sodium pertechnetate Tc 99m, Sodium phosphate P 32, Stannic pentetate Sn 117, Strontium chloride Sr 89, Technetium Tc 99m albumin, Technetium Tc 99m albumin aggregated, Technetium Tc 99m albumin colloid, Technetium Tc 99m antimony trisulfide colloid, Technetium Tc 99m apcitide, Technetium Tc 99m arcitumomab, Technetium Tc 99m bectumomab, Technetium Tc 99m biciromab, Technetium Tc 99m bicisate, Technetium Tc99m depreotide, Technetium Tc 99m disofenin, Technetium Tc 99m etidronate, Technetium Tc 99m exametazime, Technetium Tc 99m furifosmin, Technetium Tc 99m gluceptate, Technetium Tc 99m lidofenin, Technetium Tc 99m mebrofenin, Technetium Tc 99m medronate, Technetium Tc 99m mertiatide, Technetium Tc 99m nofetumomomab merpentan, Technetium Tc 99m oxidronate, Technetium Tc 99m pentetate, Technetium Tc 99m pyrophosphate, Technetium Tc 99m (pyro- and trimeta-) phosphates, Technetium Tc 99m red blood cells, Technetium Tc 99m sestamibi, Technetium Tc 99m succimer, Technetium Tc 99m sulesomab, Technetium Tc 99m sulfur colloid, Technetium Tc 99m teboroxime, Technetium Tc 99m tetrofosmin, Thallous chloride Tl 201, Water 0 15, Xenon Xe 127, or Xenon Xe 133, or 5-FDG; more preferably, the radiotracer is rubidium-82, 5-FDG, Tc-99m-sestamibi, Thallium-201 or Tc-99m-tetrophosmin.

In another aspect, the invention provides a method for monitoring myocardial perfusion abnormalities, said method comprising the steps of:
A.) administering to the subject a bolus injection, constant infusion or both of somatostatin or one or more somatostatin analogs or physiologically acceptable salts thereof,
B.) administering to the subject a detectable amount of a radiotracer in either the presence or absence of a chemical and/or physical stress agent, and
C.) obtaining an image of the subject's heart, thereby monitoring myocardial perfusion and/or myocardial viability in the subject.

Another aspect of the invention provides a kit for performing myocardial perfusion imaging comprising one or more somatostatin analogs, or physiologically acceptable salts thereof, and instructions for use. In one embodiment, the one or more somatostatin analogs are present in one or more unit dosage forms. In another embodiment, the kit further comprises tubing.

In another aspect, the invention provides a kit for performing myocardial perfusion imaging comprising
a.) a first unit dose of one or more somatostatin analogs supplied in a first vial bearing a first identification code and a second unit dose of one or more somatostatin analogs supplied in a second vial bearing a second identification code;
b.) a syringe bearing said first identification code and a syringe bearing said second identification code each independently filled with at least one pharmaceutically acceptable vehicle,
c.) at least two syringe needles; and
d.) tubing.

In one embodiment, said first unit dose of the kit is 100 mcg and said second unit dose is 200 mcg, to be administered as a 50 mcg per hour continuous infusion.

In another aspect embodiment, the invention provides a kit for performing myocardial perfusion imaging comprising
a.) a single unit dose of one or more somatostatin analogs supplied in a vial bearing an identification code;
b.) a syringe bearing said first identification code, filled with at least one pharmaceutically acceptable vehicle,
c.) a syringe needle; and
d.) tubing.

In one embodiment, the one or more somatostatin analogs is octreotide, octreotide acetate, or lanreotide.

In another embodiment, the technique to detect the presence or assess the severity of coronary artery disease or myocardial viability is radiopharmaceutical myocardial perfusion imaging.

In another embodiment, the subject is a mammal, preferably a human. In some embodiments, the human subject is a male or a female. In still other embodiments, the human subject is an elderly individual, an adult individual or an adolescent individual. In other embodiments, the human subject is suffering from one or more symptoms of coronary artery disease. In a particular embodiment, the human subject is suffering from or showing symptoms or other indications of coronary artery disease on the inferior aspect of the heart.

In another aspect, the invention provides a method for increasing the accuracy or image quality of myocardial perfusion imaging in a subject undergoing non-stress myocardial perfusion imaging comprising administering an effective amount of somatostatin or one or more somatostatin analog or a physiologically acceptable salt thereof, such that endogenous insulin release is stabilized, thereby increasing the accuracy or image quality of myocardial perfusion imaging.

In another aspect, the invention provides a method for facilitating radiotracer uptake in the heart of increasing the accuracy or image quality of myocardial perfusion imaging in a subject undergoing non-stress myocardial perfusion imaging comprising administering an effective amount of somatostatin or one or more somatostatin analog or a physiologically acceptable salt thereof, such that radiotracer uptake in the heart of said subject is facilitated, thereby increasing the accuracy or image quality of myocardial perfusion imaging.

In yet another aspect, the invention provides a method for assessing myocardial viability of a subject by non-stress myocardial perfusion imaging comprising the steps of:

a.) administering an effective amount of somatostatin or one or more somatostatin analog or a physiologically acceptable salt thereof; and

b.) obtaining an image of myocardial blood flow in the subject, thereby determining the viability of the subject myocardium

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE FIGURES

FIG. 1 shows a myocardial perfusion image of a subject's heart (seen here as a ‘doughnut’ shape) and liver using a TC99-sestamibi tracer and physiologic stress. The rest image clearly shows the liver artifact as a result of tracer uptake by the subject liver at rest. The bottom, or ‘inferior’, aspect of the heart disappears in the presence of the liver artifact. The same area of the heart reappears when the image is acquired in the absence of liver artifact (upper panel). This is an example of how liver artifact can complicate the interpretation of myocardial perfusion, especially in the inferior heart.

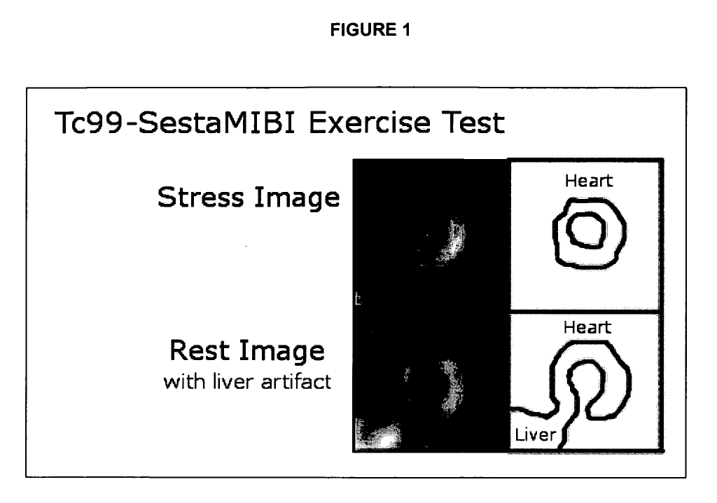

FIG. 2 shows a myocardial perfusion image of a subject's heart and liver. In this case a thallium-201 rest image (bottom row) is compared with a Tc99m-MIBI stress image (top row). Notice the high uptake in the area of the liver in the Tc99m-MIBI images which results in an artifactual absence of tracer counts from the inferior aspect of the heart. This is an example of a false-positive diagnosis of coronary stenosis due to extracardiac uptake of radiotracer (Adapted from Clin Nucl Med. 2005 September; 30(9):623-4.)

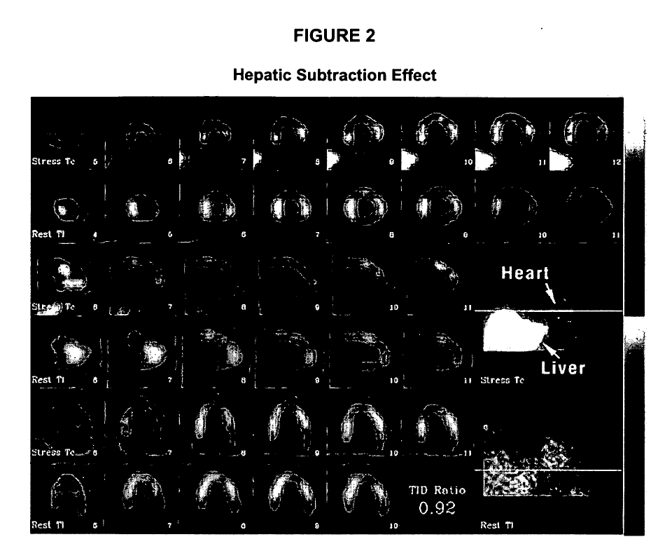

FIG. 3 shows a graph demonstrating the increased abdominal to myocardial activity ratio frequently associated with both rest and chemical stress and exercise myocardial perfusion scans. (Adapted from Clin Nucl Med. 2005 September; 30(9):623-4.)

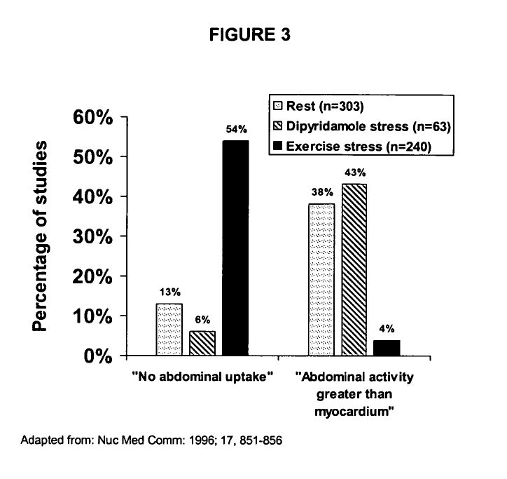

FIG. 4 shows a graph demonstrating the decrease of portal vein flow associated with the infusion of Octreotide at various infusion concentrations. (Adapted from: Digestion 1999; 60:132-140).

FIG. 5 shows a graph demonstrating a 50% reduction in gastric mucosal blood flow fifteen minutes after a single bolus injection of Octreotide as measured by a laser Doppler flowmeter. (Adapted from: Surg Endosc (2003) 17: 1570-1572).

### DETAILED DESCRIPTION

### Definitions

The following definitions can be referenced to assist in understanding the subject matter of the present application. Additional terms may be found defined throughout the detailed description.

As used herein, unless otherwise specified, the term “somatostatin” refers to a polypeptide produced by the hypothalamus and the pancreas which acts as a neurohormone that inhibits the secretion of other hormones, especially growth hormone and thyrotropin, or inhibits the secretion of the other pancreatic hormones, insulin and glucagon, and reduces the activity of the digestive system.

As used herein, unless otherwise specified, the term “somatostatin analog” refers to a somatostatin receptor agonist, which can be any naturally occurring substance or manufactured drug substance or composition that can interact and/or bind with a somatostatin receptor and initiate a biological response characteristic of the somatostatin receptor. Somatostatin. analogs include peptides having, at least about 30% sequence identity, preferably at least about 50% sequence identity, more preferably at least about 75% sequence identity or even about 80% sequence identity, still more preferably at least about 85% sequence identity or even about 90% sequence identity, even still more preferably at least about 95% sequence identity or even about 99% sequence identity with naturally occurring somatostatin. In specific embodiments, the somatostatin analog peptide is a cyclic peptide, cyclohexapeptide or an octopeptide. In other embodiments, the somatostatin analog is a small molecule. In some embodiments, somatostatin analogs include, but are not limited to, prosomatostatin, somatostatin-28 somatostatin-14, octreotide, octreotide acetate, lanreotide, seglitide, vapreotide, AN-238, RC-160, CGP 23996, BIM 23014, SMS D70, SOM 230, KE 108, L362,855, L054,522, SMS 201-995, SDZ CO611, cyclohexapeptides having somatostatin agonist properties, octopeptides having somatostatin agonist properties and small molecules having somatostatin agonist properties. In other embodiments, somatostatin analogs include those small molecules described in U.S. Pat. Nos. 6,387,932: 6,117,880; 6,063,796; 6,057,338; 6,025,372; 4,748,153; 4,663,435; 4,612,366; 4,611,054; 4,585,755; 4,522,813 4,486,415; 4,427,661; 4,360,516; 4,310,518; 4,235,886; 4,191,754; 4,190,648; 4,162,248; 4,161,521; 4,146,612; 4,140,767; 4,139,526; 4,130,554; 4,115,554; 7,094,753; 6,987,167; 6,346,601; 5,006,510; 4,130,554; 6,787,521; 6,268,342; 7,176,187; 7,060,679; 6,930,088; 6,579,967; 6,552,007; 6,465,613; 6,355,613; 6,316,414; 6,051,554; 5,998,154; 5,976,496; 5,770,687; 5,750,499; 5,597,894; 5,405,597; 5,225,180; 5,073,541; 4,428,942; and 4,393,050.

As used herein, unless otherwise specified, the term “radiotracer”, “radiotracer compound”, “radioactive tracer compound” or “radiolabelled compound” refers to a compound which is labeled with a radioactive isotope which can be detected using a camera or other device sensitive to X-rays, gamma radiation or other radiation source.

As used herein, unless otherwise specified, the term “extracardiac accumulation”, “artifact”, or “excess accumulation of a radiotracer” refers to the uptake of a radiotracer by an organ or tissue of a subject apart from and/or in addition to uptake by the myocardium. This uptake usually occurs in the liver or gut of a subject or other organ in close proximity to the heart. Extracardiac uptake decreases the diagnostic accuracy of myocardial perfusion imaging, resulting in an increase of false-positives or under-interpretation of true perfusion abnormalities.

As used herein, the term “effective amount” refers to an amount of a compound of the invention or a combination of two or more such compounds, which reduces the uptake of a radiotracer by a subject during myocardial perfusion imaging or which otherwise increases the efficiency of myocardial perfusion imaging in said subject. The amount, which is effective, will depend upon the patient's size and gender, type of image being collected, type of radiotracer being used and the result sought. For a given subject, an effective amount can be determined by methods known to those of skill in the art of myocardial perfusion imaging.

As used herein, the term “detectable amount” refers to an amount of a radiotracer or a combination of two or more such radiotracers, which is detectable by myocardial perfusion imaging using x-rays, gamma radiation or other acceptable radiation source. For a given subject, a detectable amount can be determined by methods known to those of skill in the art of myocardial perfusion imaging.

As used herein, the term “myocardial perfusion imaging” refers to radiopharmaceutical imaging performed by scintigraphy, single photon emission computed tomography (SPECT), positron emission tomography (PET), nuclear magnetic resonance (NMR) imaging, perfusion contrast echocardiography, digital subtraction angiography (DSA) and ultra fast X-ray computed tomography (CINE CT), or combinations of these techniques which allow one of skill in the art to view, analyze, and asses myocardial damage, viability or other myocardial abnormalities including, but not limited to, coronary artery disease.

As used herein, the term “physiologically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Physiologically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Physiologically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the compounds of this invention include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

As used herein, unless otherwise specified, the term “physiologically acceptable carrier,” includes, but is not limited to, a carrier medium that does not interfere with the effectiveness of the biological activity of any active ingredients, is chemically inert, and is not toxic to the consumer or patient to whom it is administered.

### Somatostatin and Somatostatin Analogs:

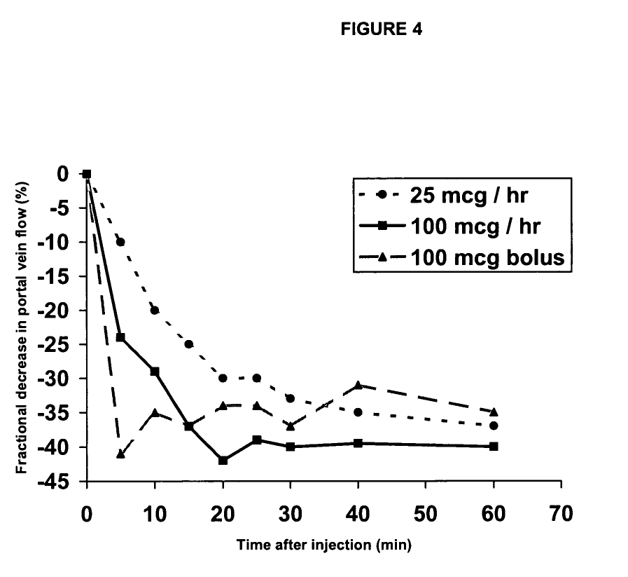

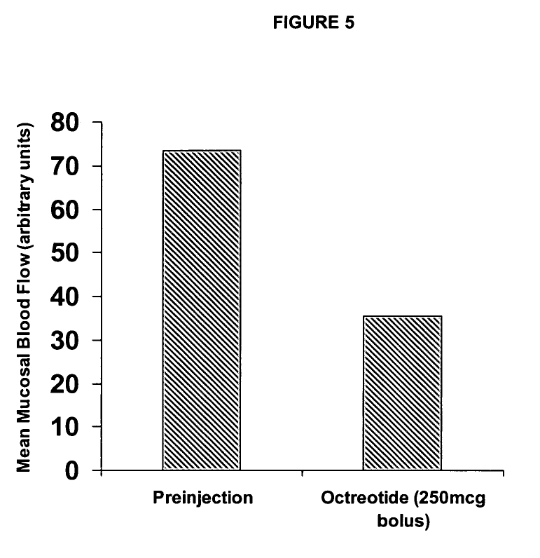

Somatostatin and its clinical analogs selectively decrease blood flow to the splanchnic viscera including the liver, small intestine and stomach. In addition, somatostatin and its clinical analogs exert suppressive effects on the release of several endogenous hormones, including insulin. (FIGS. 4-5). Somatostatin exerts these effects through a set of G-protein coupled, seven transmembrane receptors, SST₁-SST₅ (Patel, Y. C., Somatostatin and its receptor family. Front Neuroendocrinol, 1999. 20(3): p. 157-98). These receptors are broadly distributed throughout human anatomy, which accounts for the multiple physiological effects of somatostatin (Table 1). The clinically used SST agonists, octreotide and lanreotide, are selective for a subset of SST receptors, displaying affinity for SST₂, SST₃ and SST₅, but exhibiting virtually no affinity for SST₁ or SST₄.

TABLE 1

| | Distribution | | | | | | | | |
|---|---|---|---|---|---|---|---|---|---|
| | | | | | | | | | Octreotide |
| Receptor Subtype | Brain | Gut | Liver | Panc | Kidney | Lung | Aorta | Heart | EC50 |
| SST1 | + | + | + | + | + | + | + | + | >1000 |
| SST2 | + | + | + | + | + | + | + | + | 0.6 |
| SST3 | + | + | | + | | | + | + | 34.5 |
| SST4 | + | + | | + | | + | + | + | >1000 |
| SST5 | + | + | | + | | | + | + | 7 |

Affinities are expressed in EC50 (nM). Data adapted from Patel, 1999 (Patel, Y. C., Somatostatin and its receptor family. Front Neuroendocrinol, 1999. 20(3): p. 157-98).
nd = not done.

The diagnostic accuracy of myocardial perfusion imaging relies in part on the absence of artifact, and in part on the predictable response of the heart to either exercise or pharmacologically induced coronary vasodilation. Without being limited by theory, the administration of somatostatin analogs before and during MPI is believed to reduce artifact and/or extracardiac uptake by a subject without altering the basic coronary vasodilatory response to common stress agents, including chemical agents adenosine and dipyridamole, and without significantly effecting the systolic or diastolic parameters of baseline cardiac function.

It is also recognized that, in the setting of myocardial viability assessment using the combination of 5-FDG radiotracer and PET scanning, an additional benefit of periprocedural somatostatin analog administration will include an inhibitory effect on endogenous insulin secretion. By inhibiting endogenous insulin secretion, somatostatin analog administration will allow for easier control of serum glucose levels using exogenous insulin and therefore aid in a controlled myocardial uptake of the radiotracer, 5-FDG, which is a glucose analog.

### Dosage Forms and Modes of Administration

Preferred modes of administration include oral administration and parenteral administration, including but not limited to bolus injection and constant infusion. More preferred modes of administration include bolus injection and constant infusion either alone or in combination with each other.

### Oral Dosage Forms

Somatostatin or Somatostatin analogs of the invention and compositions comprising them that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent very advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103.TM and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Parenteral Dosage Forms

Parenteral dosage forms can be administered to patients by various routes including, but not limited to, subcutaneous, intravenous (including bolus injection and constant infusion), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products (including, but not limited to lyophilized powders, pellets, and tablets) ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Dosage

The magnitude of the effective dose of somatostatin or one or more somatostatin analogs or physiologically salts thereof in the reduction in splanchnic blood flow at the time of radiotracer injection will vary with the severity of the toxicity and the route of administration. The dose, and perhaps the dose frequency, will also vary according to age, body weight, response, and the past medical history of the subject and radiotracer and type of radiation being used in a given myocardial perfusion imaging study. Suitable dosing regimens can be readily selected by those skilled in the art with due consideration of such factors. All combinations described in the specification are encompassed as therapeutic, and it is understood that one of skill in the art would be able to determine a proper dosage of particular somatostatin analog and radiotracer using the parameters provided in the invention.

In general, the total daily dose ranges of somatostatin or the somatostatin analog are generally from about 0.02 mcg/kg to about 10 mcg/kg administered in bolus injection or about 0.02 mcg/kg/hr to about 0.4 mcg/kg/hr administered as a constant infusion. A preferred total dose is from about 20 mcg to about 700 mcg of somatostatin or the somatostatin analog per bolus injection, more preferably about 25 mcg to about 250 mcg, even more preferably about 50 mcg to about 200 mcg, still more preferably about 50 mcg to about 100 mcg. Similarly, a preferred total rate dose for constant infusion is from about 20 mcg/hr to about 400 mcg/hr of somatostatin or the somatostatin analog per infusion, more preferably about 50 mcg/hr to about 250 mcg/hr, even more preferably about 50 mcg/hr to about 150 mcg/hr, still more preferably about 100 mcg/hr.

The total daily dose ranges of somatostatin or the somatostatin analog when administered orally generally range from about 0.001 mg/kg to about 200 mg/kg body weight per day, and preferably from about 0.01 mg/kg to about 20 mg/kg body weight per day.

Alternatively, somatostatin or the somatostatin analog or combination of somatostatin analogs is given as a single bolus injection, followed by a constant infusion. Preferably, somatostatin or the somatostatin analog or combination of somatostatin analogs is given as a single bolus injection up to about 6 hours before administration of the radiotracer, more preferably up to about 2 hours before, even more preferably up to about 1 hour before, still more preferably between about 5 minutes and 30 minutes before. Preferably, the single bolus injection is followed by a constant infusion of the somatostatin analog or combination of somatostatin analogs, which may be the same or different as the analog or analogs administered via bolus injection. Preferably, the constant infusion is begun at the same time as the bolus injection, more preferably from about 15 minutes to about 2 hours before administration of the radiotracer, still more preferably about 5 minutes to about 30 minutes before the administration of the radiotracer. Preferably, the constant infusion is ceased after the accumulation of the final myocardial perfusion imaging scans, more preferably from about 15 minutes to about 2 hours before or after accumulation of the final myocardial perfusion imaging scans. As the somatostatin analogs are not particularly toxic, the formulation may be administered for as long as necessary to achieve the desired effect.

Alternatively still, when administered orally, somatostatin or the somatostatin analog or combination of somatostatin analogs is given up to about 36 hours before administration of the radiotracer, more preferably up to 24 hours before, even more preferably up to about 12 hours before, yet more preferably up to about 6 hours before, still more preferably between about 15 minutes before and about 3 hours before.

### Imaging Methods

Suitable myocardial perfusion imaging studies can be performed by those of skill in the art of radiology and radioimaging in accordance with generally accepted practices. The myocardial perfusion imaging study, including the source or type of radiation, imaging system and data collection system will vary according to age, body weight, response, and past medical history of the subject. Suitable myocardial perfusion imaging studies can be readily selected by those skilled in the art with due consideration of such factors.

In general, suitable myocardial perfusion imaging studies can be performed by scintigraphy, single photon emission computed tomography (SPECT), positron emission tomography (PET), nuclear magnetic resonance (NMR) imaging, perfusion contrast echocardiography, digital subtraction angiography (DSA) and ultra fast X-ray computed tomography (CINE CT), or combinations of these techniques.

### Kits

Typically, active ingredients of the invention are administered to a subject prior to and/or during a myocardial perfusion imaging study. In addition, active ingredients of the invention are administered prior to and/or simultaneously with a radiotracer. This invention therefore encompasses kits which, when used by the medical practitioner, can simplify the administration of appropriate amounts of active ingredients and/or radiotracers to a patient.

A typical kit of the invention comprises one or more unit dosage forms of somatostatin or one or more somatostatin analogs, or physiologically acceptable salts thereof, and instructions for use. A kit of the invention may also further comprise a unit dosage form of a radiotracer. Examples of radiotracers include, but are not limited to, those listed above.

Kits of the invention can further comprise devices that are used to administer somatostatin or the somatostatin analog and/or radiotracer. Examples of such devices include, but are not limited to, intravenous cannulation devices, syringes, drip bags, patches, topical gels, pumps, tubing, containers that provide protection from photodegredation, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Kits of the invention can further compromise devices and methods that facilitate the simultaneous administration of somatostatin or one or more somatostatin analogs with chemical stress agents such as, but not limited to, adenosine, A2a receptor agonists, dipyridamole or dobutamine. Examples include but are not limited to multiple ports on supplied tubing, salt derivatives of one or more somatostatin analogs designed to be compatible in intravenous delivery tubing with common chemical stress agents or timing devices that automatically switch from one agent to another.

The invention will now be further described by way of the following non-limiting examples.

### EXAMPLES

### Myocardial Perfusion Imaging Using Octreotide and Pharmacologic Stress

Fifteen minutes prior to injection of a radiotracer, a bolus injection of 100 mcg octreotide is administered, followed by a constant infusion of 100 mcg per hour for the remainder of the study including rest and stress injections. Adenosine pharmacologic stress is administered as normal without suspension of octreotide. After the final perfusion scan is acquired, octreotide is turned off.

Analysis of myocardial perfusion imaging study is performed as usual except extracardiac uptake of radiotracer as a result of reduced splanchic blood flow are reduced and efficacy of analysis is improved.

### Myocardial Perfusion Imaging Using Octreotide and Pharmacologic Stress

Ten minutes prior to injection of a radiotracer, a bolus injection of 100 mcg octreotide is administered, rest perfusion images are acquired as normal. Ten minutes prior to adenosine pharmacologic stress, a second 100 mcg octreotide bolus is administered and the stress perfusion scan is acquired as normal.

Analysis of myocardial perfusion imaging study is performed as usual except extracardiac uptake of radiotracer as a result of reduced of splanchic blood flow are reduced and efficacy of analysis is improved.

### Myocardial Perfusion Imaging Using Lanreotide and Physiologic Stress

Thirty minutes prior to injection of a radiotracer, a bolus injection of 200 mcg lanreotide is administered, followed by a constant infusion of 200 mcg per hour for the remainder of the rest injections. Prior to administration of physiologic treadmill exercise stress, lanreotide is turned off.

Analysis of myocardial perfusion imaging study is performed as usual except extracardiac uptake of radiotracer as a result of reduced of splanchic blood flow are reduced and efficacy of analysis is improved.

### Kit Designed to Facilitate the Delivery of Periprocedural Octreotide During Myocardial Perfusion Imaging

Unit dosages of 100 mcg octreotide acetate and 200 mcg octreotide acetate are supplied in two separate vial containers of different colors. Two diluent filled syringes, each color coded to the appropriate vial are supplied along with two 1½″ syringe needles, allowing for easy reconstitution of octreotide. A single vial with 200 mcg is reconstituted with 50 cc D5W and supplied tubing is attached to this vial and octreotide drip is begun at the specified rate. In the second vial, 100 mcg octreotide is reconstituted with supplied syringe and needle. Solution is withdrawn and given intravenously as a bolus administration through an auxiliary port in the supplied tubing. All instructions are supplied. Chemical stress agent and radio tracer are then administered through same auxiliary port.

### Myocardial Perfusion Imaging Comparative Trial

A Randomized, placebo-controlled, double blinded trial for the use of octreotide acetate to suppress subdiaphragmatic uptake in myocardial perfusion imaging is performed.

40 subjects with recent history of dipyridamole or adenosine myocardial perfusion scan are enrolled. 20 of these subjects have anterior or lateral reversibility.

The subjects are initially randomized into equal groups, one to receive octreotide, the other to receive placebo.

a. Treatment group: 100 mcg octreotide acetate is infused as bolus injection 15 minutes prior to rest images. This is followed by a 100 mcg/hr constant infusion for the remainder of the study.

b. Placebo group: Equivalent volume of saline is infused at identical rate as treatment group.

Blood pressure, heart rate and symptoms are monitored in the standard fashion. Perfusion agent injection and rest images are performed in the standard fashion. Dipyridamole or adenosine stress is infused and stress images acquired in the standard fashion. Octreotide or saline infusion is halted after completion of each imaging study.

Imaging scans are analyzed in the standard fashion. Subdiaphragmatic uptake is rated subjectively as “not present”, “insignificant” or “obscuring” by the reader and a quantitative subdiaphragmatic uptake score is generated. Perfusion study data is read and compared to previous study by three independent readers and the effect of treatment on obscuring artifact is calculated along with the effect on revision of past interpretation of the scan.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### REFERENCES

1. Pawlikowski, M. and G. Melen-Mucha, Somatostatin analogs—from new molecules to new applications. Curr Opin Pharmacol, 2004. 4(6): p. 608-13.
2. Heart Disease and Stroke Statistics-2006 Update. 2006, American Heart Association: Dallas, Tex. http://www.americanheart.org.
3. World Health Report 1997: Conquering Suffering, Enriching Humanity. 1997, World Health Organization: Geneva, http://www.who.int/whr/1997/en/. p. 168.
4. World Health Report 2002: Reducing risks, promoting healthy life. 2002, Geneva, World Health Organization: http://www.who.int/whr/2002/en/. p. 256.
5. Klocke F J, B. M., Bateman T M, Berman D S, Carabello B A, Cerqueira M D, DeMaria A N, Kennedy J W, Lorell B H, Messer J V, O'Gara P T, Russel R O Jr., St. John Sutton, M G, Udelson J E, Verani M S, Williams K A., ACC/AHA/ASNC Guidelines for the Clinical Use of Cardiac Radionuclide Imaging: A report of the American College of Cardiology/American Heart Association Task Force on Practice Guidelines. 2003, American College of Cardiology/American Heart Association: Bethesda, Md. p. 69.
6. Zipes, D., Libby, P., Bonow, R., Braunwald, E., Braunwald's Heart Disease: A Textbook of Cardiovascular Medicine. 7th ed. 2005, Philadelphia: Elsevier Saunders. 2183.
7. Medicare Part B Payments for Myocardial Perfusion Imaging, J. Rehnquist, Editor. 2002, Department of Health and Human Services, Office of the Inspector General. p. 2.
8. Mitka, M., Costly surge in diagnostic imaging spurs debate. Jama, 2005. 293(6): p. 665-7.
9. Levin, D. C., et al., Recent rapid increase in utilization of radionuclide myocardial perfusion imaging and related procedures: 1996-1998 practice patterns. Radiology, 2002. 222(1): p. 144-8.
10. Robert C Hendel, P., American Society of Nuclear Cardiology. 2004 ACC address quoting the ‘Nuclear Medicine Market Summary Report 2003, Des Plaines Ill.: IMV Medical Information Division. ’
11. Cardiolite Product Insert. 2003, Bristol-Myers Squibb, Medical Imaging: N. Billerica, MA. p. 2.
12. Hurwitz, G. A., Increased extra-cardiac background uptake on immediate and delayed post-stress images with 99Tcm sestamibi: determinants, independence, and significance of counts in lung, abdomen and myocardium. Nucl Med Commun, 2000. 21(10): p. 887-95.
13. Rehm, P. K., et al., Frequency of extra-cardiac activity and its effect on 99Tcm-MIBI cardiac SPET interpretation. Nucl Med Commun, 1996. 17(10): p. 851-6.
14. Heller, E. N., et al., Extracardiac activity complicates quantitative cardiac SPECT imaging using a simultaneous transmission-emission approach. J Nucl Med, 1997. 38(12): p. 1882-90.
15. Germano, G., et al., A quantitative phantom analysis of artifacts due to hepatic activity in technetium-99m myocardial perfusion SPECT studies. J Nucl Med, 1994. 35(2): p. 356-9.
16. Ohnishi, H., et al., [A phantom study for the evaluation of the effect of the high uptake in the liver on technetium-99m myocardial perfusion SPECT images]. Kaku Igaku, 1998. 35(6): p. 375-83.
17. King, M. A., et al., A Monte Carlo investigation of artifacts caused by liver uptake in single photon emission computed tomography perfusion imaging with technetium 99m-labeled agents. J Nucl Cardiol, 1996. 3(1): p. 18-29.
18. Hurwitz, G. A., et al., The VEX-test for myocardial scintigraphy with thallium-201 and sestamibi: effect on abdominal background activity. J Nucl Med, 1995. 36(6): p. 914-20.
19. Middleton, G. W. and J. H. Williams, Significant gastric reflux of technetium-99m-MIBI in SPECT myocardial imaging. J Nucl Med, 1994. 35(4): p. 619-20.
20. Nuyts, J., et al., A study of the liver-heart artifact in emission tomography. J Nucl Med, 1995. 36(1): p. 133-9.
21. Bai, J., et al., Comparison of image reconstruction algorithms in myocardial perfusion scintigraphy. Ann Nucl Med, 2001. 15(1): p. 79-83.
22. Iskandrian, A. E., Verani, Mario S., Nuclear Cardiac Imaging: Principles and Applications. 2003, New York, N.Y.: Oxford Press. 509.
23. Dogruca, Z., et al., A comparison of Tl-201 stress-reinjection-prone SPECT and Tc-99m-sestamibi gated SPECT in the differentiation of inferior wall defects from artifacts. Nucl Med Commun, 2000. 21(8): p. 719-27.
24. Schoss, R. M. and R. J. Gorten, Comparison of supine versus prone tomographic myocardial imaging. Effect on false positive rate. Clin Nucl Med, 1996. 21(6): p. 445-51.
25. Kiat, H., et al., Quantitative stress-redistribution thallium-201 SPECT using prone imaging: methodologic development and validation. J Nucl Med, 1992. 33(8): p. 1509-15.
26. Segall, G. M., M. J. Davis, and M. L. Goris, Improved specificity of prone versus supine thallium SPECT imaging. Clin Nucl Med, 1988. 13(12): p. 915-6.
27. Esquerre, J. P., Prone versus supine thallium-201 myocardial SPECT. J Nucl Med, 1989. 30(10): p. 1738-9.
28. Segall, G. M. and M. J. Davis, Prone versus supine thallium myocardial SPECT: a method to decrease artifactual inferior wall defects. J Nucl Med, 1989. 30(4): p. 548-55.
29. Boz, A., et al., The volume effect of the stomach on intestinal activity on same-day exercise—rest Tc-99m tetrofosmin myocardial imaging. Clin Nucl Med, 2001. 26(7): p. 622-5.
30. Boz, A., et al., The effects of solid food in prevention of intestinal activity in Tc-99m tetrofosmin myocardial perfusion scintigraphy. J Nucl Cardiol, 2003. 10(2): p. 161-7.
31. Hurwitz, G. A., et al., Investigation of measures to reduce interfering abdominal activity on rest myocardial images with Tc-99m sestamibi. Clin Nucl Med, 1993. 18(9): p. 735-41.
32. van Dongen, A. J. and P. P. van Rijk, Minimizing liver, bowel, and gastric activity in myocardial perfusion SPECT. J Nucl Med, 2000. 41(8): p. 1315-7.
33. Weinmann, P. and J. L. Moretti, Metoclopramide has no effect on abdominal activity of sestamibi in myocardial SPET. Nucl Med Commun, 1999. 20(7): p. 623-5.
34. Yuksel, M., et al., The effect of aminophylline administration on 99 mTc-MIBI lung and liver uptake in patients with or without myocardial ischemia. Rev Esp Med Nucl, 2000. 19(6): p. 423-7.
35. Reichlin, S., Somatostatin. N Engl J Med, 1983. 309(24): p. 1495-501.
36. Reichlin, S., Somatostatin (second of two parts). N Engl J Med, 1983. 309(25): p. 1556-63.
37. Lamberts, S. W., et al., Octreotide. N Engl J Med, 1996. 334(4): p. 246-54.
38. Sandostatin Product Insert. 2002, Novartis: Stein, Switzerland. p. 12.
39. Rosenoff, S. H., et al., A multicenter, randomized trial of long-acting octreotide for the optimum prevention of chemotherapy-induced diarrhea: results of the STOP trial. J Support Oncol, 2006. 4(6): p. 289-94.
40. Gotzsche, P. C. and A. Hrobjartsson, Somatostatin analogs for acute bleeding oesophageal varices. Cochrane Database Syst Rev, 2005(1): p. CD000193.
41. Sonnenberg, G. E., et al., Effect of somatostatin on splanchnic hemodynamics in patients with cirrhosis of the liver and in normal subjects. Gastroenterology, 1981. 80(3): p. 526-32.
42. Schoemaker, R. C., J. Burggraaf, and A. F. Cohen, Assessment of hepatic blood flow using continuous infusion of high clearance drugs. Br J Clin Pharmacol, 1998. 45(5): p. 463-9.
43. Burggraaf, J., et al., Sorbitol as a marker for drug-induced decreases of variable duration in liver blood flow in healthy volunteers. Eur J Pharm Sci, 2000. 12(2): p. 133-9.
44. Clarke, D. L., A. McKune, and S. R. Thomson, Octreotide lowers gastric mucosal blood flow in normal and portal hypertensive stomachs. Surg Endosc, 2003. 17(10): p. 1570-2.
45. Kubba, A. K., et al., The effect of octreotide on gastroduodenal blood flow measured by laser Doppler flowmetry in rabbits and man. Am J Gastroenterol, 1999. 94(4): p. 1077-82.
46. Schiedermaier, P., et al., Effects of different octreotide dosages on splanchnic hemodynamics and glucagon in healthy volunteers. Digestion, 1999. 60(2): p. 132-40.
47. Wahren, J. and L. S. Eriksson, The influence of a long-acting somatostatin analog on splanchnic hemodynamics and metabolism in healthy subjects and patients with liver cirrhosis. Scand J Gastroenterol Suppl, 1986. 119: p. 103-8.
48. Eriksson, L. S, and J. Wahren, Intravenous and subcutaneous administration of a long-acting somatostatin analog: effects on glucose metabolism and splanchnic hemodynamics in healthy subjects. Eur J Clin Invest, 1989. 19(2): p. 213-9.
49. McCormick, P. A., et al., Superior mesenteric artery blood flow in man measured with intra-arterial Doppler catheters: effect of octreotide. J Hepatol, 1993. 17(1): p. 20-7.
50. Cooper, A. M., et al., Fasting and post-prandial splanchnic blood flow is reduced by a somatostatin analog (octreotide) in man. Clin Sci (Lond), 1991. 81(2): p. 169-75.
51. Patel, Y. C., Somatostatin and its receptor family. Front Neuroendocrinol, 1999. 20(3): p. 157-98.
52. Smith, W. H., et al., Somatostatin receptor subtype expression in the human heart: differential expression by myocytes and fibroblasts. J Endocrinol, 2005. 187(3): p. 379-86.
53. Granata, R., et al., H9c2 cardiac muscle cells express all somatostatin receptor subtypes. J Endocrinol Invest, 2004. 27(9): p. RC24-7.
54. Badway, A. C., et al., Somatostatin regulates intracellular signaling in human carotid endothelial cells. Biochem Biophys Res Commun, 2004. 319(4): p. 1222-7.
55. Capaldo, B., et al., Acute hyperglycemia does not affect the reactivity of coronary microcirculation in humans. J Clin Endocrinol Metab, 2005. 90(7): p. 3871-6.
56. Tyden, G., et al., The effect of somatostatin on blood flow through coronary bypass grafts in anesthetized man. Acta Chir Scand, 1981. 147(5): p. 331-3.
57. Law, W. R. and M. P. McLane, Adenosine enhances myocardial glucose uptake only in the presence of insulin. Metabolism, 1991. 40(9): p. 947-52.
58. Erbas, T., et al., Short-term effects of somatostatin analog (SMS 201-995) on left ventricular function in healthy persons: a scintigraphic study. J Endocrinol Invest, 1993. 16(11): p. 857-61.
59. Colao, A., et al., Reversal of acromegalic cardiomyopathy in young but not in middle-aged patients after 12 months of treatment with the depot long-acting somatostatin analog octreotide. Clin Endocrinol (Oxf), 2003. 58(2): p. 169-76.
60. Garland, J., et al., Sandostatin LAR (long-acting octreotide acetate) for malignant carcinoid syndrome: a 3-year experience. Aliment Pharmacol Ther, 2003. 17(3): p. 437-44.
61. McCormick, P. A., et al., Cardiovascular effects of octreotide in patients with hepatic cirrhosis. Hepatology, 1995. 21(5): p. 1255-60.
62. Dilger, J. A., et al., Octreotide-induced bradycardia and heart block during surgical resection of a carcinoid tumor. Anesth Analg, 2004. 98(2): p. 318-20, table of contents.
63. Herrington, A. M., K. W. George, and C. C. Moulds, Octreotide-induced bradycardia. Pharmacotherapy, 1998. 18(2): p. 413-6.
64. Schiedermaier, P., B. Goke, and T. Sauerbruch, Effects of different octreotide dosages on splanchnic hemodynamics and glucagon in patients with TIPS. Am J Gastroenterol, 2001. 96(7): p. 2218-24.

1. A method for reducing extracardiac accumulation of a radiotracer during myocardial perfusion imaging in a subject comprising administering to a subject in need of myocardial perfusion imaging an effective amount of somatostatin or one or more somatostatin analogs or a physiologically acceptable salt thereof, thereby reducing extracardiac accumulation of a radiotracer during myocardial perfusion imaging.

2. The method of claim 1, wherein the somatostatin analog is prosomatostatin, somatostatin-28, somatostatin-14, octreotide, octreotide acetate, lanreotide, seglitide, vapreotide, AN-238, SMS 201-995, SDZ CO 611, RC 160, SMS-D70, SOM 230, KE 108, CGP 23996, BIM 23014, L362,855, or L054,522.

3. The method of claim 2, wherein the somatostatin analog is octreotide, octreotide acetate, or lanreotide.

4. The method of claim 1, wherein the myocardial perfusion imaging is stress myocardial perfusion imaging.

5. The method of claim 4, wherein the stress myocardial perfusion imaging utilizes a chemical stress agent.

6. The method of claim 5, wherein the chemical stress agent is adenosine, dipyridamole, dobutamine, or an A2a receptor agonist.

7. The method of claim 1, further comprising administering one or more additional somatostatin analogs or physiologically acceptable salts thereof.

8. The method of claim 1, wherein the subject is a mammal.

9. The method of claim 7, wherein the mammal is a human.

10. The method of claim 1, wherein somatostatin or the somatostatin analog is administered: orally; as one or more bolus injections; or via constant infusion.

11. (canceled)

12. (canceled)

13. The method of claim 1, wherein somatostatin or the somatostatin analog is administered: prior to the administration of the radiotracer; or simultaneously with as the radiotracer.

14. (canceled)

15. The method of claim 1, wherein somatostatin or the somatostatin analog is administered as a single bolus injection prior to the administration of the radiotracer followed by a constant infusion the somatostatin analog or an additional somatostatin analog.

16. The method of claim 10, wherein the effective amount is about 0.2 mcg/kg to about 10 mcg/kg.

17. The method of claim 11, wherein the effective amount is about 0.2 mcg/kg/hr to about 10 mcg/kg/hr.

18. The method of claim 15, wherein the effective amount is about 0.02 mcg/kg to about 10 mcg/kg administered as a bolus injection followed by about 0.02 mcg/kg/hr to about 10 mcg/kg/hr administered as a constant infusion.

19. The method of claim 1, wherein the radiotracer is rubidium-82, 5-FDG, Tc-99m-sestamibi, Thallium-201 or Tc-99m-tetrophosmin.

20. A method for monitoring myocardial perfusion abnormalities in a subject, said method comprising the steps of:
a.) administering to the subject a bolus injection, constant infusion or both of somatostatin or one or more somatostatin analogs or physiologically acceptable salts thereof,
b.) administering to the subject a detectable amount of a radiotracer in either the presence or absence of a chemical stress agent, and
c.) obtaining an image of myocardial blood flow in the subject, thereby monitoring myocardial perfusion abnormalities in the subject.

21. (canceled)

22. A kit for performing myocardial perfusion imaging comprising somatostatin or one or more somatostatin analogs, or physiologically acceptable salts thereof and instructions for use.

23-26. (canceled)

27. The kit of claim 22 comprising
a first unit dose of one or more somatostatin analogs supplied in a first vial bearing a first identification code and a second unit dose of one or more somatostatin analogs supplied in a second vial bearing a second identification code;
a syringe bearing said first identification code and a syringe bearing said second identification code each independently filled with at least one pharmaceutically acceptable vehicle,
at least two syringe needles; and
tubing.

28. (canceled)

29. (canceled)

30. A method for increasing the accuracy or image quality of myocardial perfusion imaging in a subject undergoing non-stress myocardial perfusion imaging comprising administering an effective amount of somatostatin or one or more somatostatin analog or a physiologically acceptable salt thereof, such that:
a.) endogenous insulin release is stabilized; or
b.) radiotracer uptake in the heart of said subject is facilitated; thereby increasing the accuracy or image quality of myocardial perfusion imaging.

31. (canceled)

32. A method for assessing myocardial viability of a subject by non-stress myocardial perfusion imaging comprising the steps of:
a.) administering an effective amount of somatostatin or one or more somatostatin analog or a physiologically acceptable salt thereof; and
b.) obtaining an image of myocardial blood flow in the subject, thereby determining the viability of the subject myocardium.

33. (canceled)

34. (canceled)
